(19) United States
(12) Patent Application Publication
(10) Pub. No.: US 2026/0244619 A1
(43) Pub. Date: Aug. 20, 2026

(54) METHOD AND SYSTEM FOR ANSWERING QUERY USING TEXT-TO-SQL MODEL FOR ELECTRONIC HEALTH RECORD

(71) Applicant: LG MANAGEMENT DEVELOPMENT INSTITUTE CO., LTD., Seoul (KR)

(72) Inventor: Yong Rae JO, Seoul (KR)

(21) Appl. No.: 19/641,465

(22) Filed: Apr. 7, 2026

Related U.S. Application Data
(63) Continuation of application No. PCT/KR2025/006682, filed on May 16, 2025.

(30) Foreign Application Priority Data
Jun. 13, 2024 (KR) 10-2024-0077226
Feb. 26, 2025 (KR) 10-2025-0025302

(51) Int. Cl. G06F 16/2452; G06F 16/242; G06N 3/0895; G16H 10/60

(52) U.S. Cl. CPC G06F 16/24522; G06F 16/243; G06N 3/0895; G16H 10/60

(57) ABSTRACT

A method for answering a query using a text-to-SQL (Structured Query Language) model for an electronic health record performed by a computing device comprises: receiving a natural language query from a user; generating an output value including one of an SQL query and a null value for the natural language query through the text-to-SQL model configured to process the natural language query; and providing the user with a response result for the natural language query based on the output value.

### CROSS-REFERENCE TO RELATED APPLICATIONS

This application is a continuation of International Patent Application No. PCT/KR2025/006682, filed on May 16, 2025, which claims the benefit of and priority to Korean Patent Application No. 10-2024-0077226, filed on Jun. 13, 2024, and Korean Patent Application No. 10-2025-0025302, filed on Feb. 26, 2025, the entire disclosures of which are hereby incorporated herein by reference in their entireties.

### BACKGROUND

### Technical Field

The present disclosure generally relates to a method and a system for answering a query using a text-to-SQL (Structured Query Language) model for an electronic health record. More specifically, some embodiments of the present disclosure relate to a method and a system for answering a query by providing an appropriate response result to a user query using a text-to-SQL model for an electronic health record constructed based on multi-stage self-training and data filtering.

### Related Art

An electronic health record (EHR) is a medical database that comprehensively manages various clinical information such as patient diagnosis, treatment, medication, surgery, and discharge, and plays an essential role in a clinical field.

However, access to the EHR generally requires knowledge of query languages such as SQL, which has served as a significant entry barrier for medical professionals.

For example, even when a clinical expert such as a doctor or a nurse wants to quickly retrieve patient information, it may be difficult to use the EHR system without knowledge of SQL syntax, which frequently results in wasted time and manpower in an urgent medical situation.

In one example, in a medical field, an artificial intelligence (AI) technology is already widely used in patient treatment, clinical research, and administrative efficiency improvement, and is bringing innovation in various areas, from customized treatment recommendations to early disease detection based on its ability to analyze large-scale data.

In this trend, a text-to-SQL model that converts natural language queries into SQL queries is emerging, and a useful technology because the text-to-SQL can support the medical professionals to utilize the electronic health record without SQL knowledge.

In particular, with the development of a large language model (LLM), an ability to accurately translate complex medical inquiries into the SQL queries is also steadily improving.

However, to actually apply the text-to-SQL model in a medical environment, reliability beyond simple query conversion capability is required.

For example, when the SQL query is forcedly generated even when a query is ambiguous or the model does not know a correct answer, it may cause a fatal error in a decision-making process.

Provision of misinformation in the medical field is likely to pose a direct threat to patient safety, and is also able to negatively affect various clinical research and administrative tasks.

Therefore, it is an essential task for the text-to-SQL model to actively identify ‘unanswerable queries’ and to have a mechanism that avoids incorrect answers or requires additional verification processes therefor.

### SUMMARY

According to an embodiment of the present disclosure, a text-to-SQL model for an electronic health record (EHR) may be constructed based on multi-stage self-training and data filtering, and an appropriate response result to a user query using the same may be provided.

However, technical purposes to be achieved by embodiments of the present disclosure are not limited to the technical purposes described above, and other technical purposes may exist.

A method for answering a query using a text-to-SQL model for an electronic health record performed by a computing device according to an embodiment of the present disclosure comprises: receiving a natural language query from a user; generating an output value including one of an SQL query and a null value for the natural language query through the text-to-SQL model configured to process the natural language query; and providing the user with a response result for the natural language query based on the output value.

In another aspect, the providing the user with the response result may include providing the user with data retrieved from an EHR database storing therein the electronic health record based on the SQL query when the output value includes the SQL query.

In another aspect, the providing the user with the response result may include providing at least one of a query rejection response and a response suggesting an additional confirmation procedure to the user when the output value includes the null value.

In another aspect, wherein the response suggesting the additional confirmation procedure to the user may include at least one of a request to rephrase the query for clarification, a suggestion to search external information, and a guidance to contact a system administrator. The external information may refer to a supplementary data source located outside a primary database (EHR) referenced by the text-to-SQL model, and may include, for example, an authorized medical database, medical-related content from an external website, or a digital document external to the system, and the like.

In another aspect, the generating of the output value may include generating the output value using the text-to-SQL model that has performed one of multi-stage self-training of re-training itself based on an output value generated by itself after initial training, and prediction result filtering.

In another aspect, the multi-stage self-training process may include: setting a model data environment including the EHR database storing therein the electronic health record and an EHRSQL database storing an EHRSQL; training a seed model configured to process a predetermined natural language query based on the EHR database and an EHRSQL database-based training data set; obtaining pseudo-labeling data that is data assigned with a predetermined pseudo-label, based on a seed fine-tuned model that is the trained seed model; augmenting the training data set based on the pseudo-labeling data; and re-training the seed model based on the augmented training data set.

In another aspect, the EHRSQL may include a predetermined SQL query based on the predetermined natural language query and a schema of the EHR database.

In another aspect, the EHRSQL database may include the training data set that is a data set used for model training and a test data set that is a data set used for model evaluation.

In another aspect, the obtaining of the pseudo-labeling data may include obtaining an output value for each of test sample data included in the test data set based on the seed fine-tuned model.

In another aspect, the obtaining of the pseudo-labeling data may include mapping, when the obtained output value is null, a pseudo-label having a form corresponding to the null to test sample data having an output of null.

In another aspect, the prediction result filtering may include at least one of maximum entropy filtering and execution-based filtering.

In addition, a system for answering a query using a text-to-SQL model for an electronic health record according to an embodiment of the present disclosure comprise: at least one memory; and at least one processor configured to read at least one application stored in the memory and perform a method for answering the query using the text-to-SQL model for the electronic health record, wherein instructions of the processor include instructions for: receiving a natural language query from a user; generating an output value including one of an SQL query and a null value for the natural language query through the text-to-SQL model configured to process the natural language query; and providing the user with a response result for the natural language query based on the output value.

In another aspect, the system may further comprise a plurality of neurons configured in an array including at least one register, at least one programmable logic, and at least one input interface; a plurality of synaptic circuits configured to store therein synaptic weights for adjusting connection strengths among the plurality of neurons; and at least one routing network configured to control a data flow between the plurality of neurons, wherein each of the plurality of neurons further includes a field programmable gate array (FPGA) implementation for a predetermined artificial neural network connected to at least other neuron through the routing network to set a transmission path of the weights.

In another aspect, the system may further comprise a plurality of neurons organized in an array including at least one register, at least one microprocessor, and at least one input; and a plurality of synaptic circuits configured to store therein synaptic weights for adjusting connection strengths among the plurality of neurons, wherein each of the plurality of neurons further includes an application specific integrated circuit (ASIC) for a predetermined artificial neural network connected to at least other neuron through one of the plurality of synaptic circuits.

A method and system for answering a query using a text-to-SQL model for an electronic health record according to an embodiment of the present disclosure may construct the text-to-SQL model for the electronic health record based on multi-stage self-training and data filtering, and use the constructed text-to-SQL model, thereby allowing the user to safely retrieve accurate electronic health record data without SQL knowledge, and thus, minimizing the propagation of incorrect information and further improving accuracy of the medical decision making and the patient treatment efficiency.

A method and system for answering a query using A text-to-SQL model for an electronic health record according to an embodiment of the present disclosure may provide a text-to-SQL solution that lowers the barrier to entry caused by the need to acquire specialized knowledge regarding SQL and enhances reliability, thereby providing a practical solution for realizing a stable and rapidly-implemented query answering in an electronic health record environment and reducing the gap between a medical technology and clinical practice.

In addition, a method and system for answering a query using a text-to-SQL model for an electronic health record according to an embodiment of the present disclosure may automatically assign a pseudo-label for an unanswerable query to the text-to-SQL model through a multi-stage self-training technique and support an additional training based on an extended data set including the pseudo-label, thereby providing the text-to-SQL model with the improved performance that stably generates the SQL query with the higher accuracy and reliability in the actual medical environment.

In addition, a method and system for answering a query using a text-to-SQL model for the electronic health record according to an embodiment of the present disclosure may allow the text-to-SQL model for the electronic health record to more accurately detect the unanswerable query or uncertain prediction, and to refuse to provide the answer therefor or perform an additional verification procedure.

Additionally, a method and system for answering a query using a text-to-SQL model for an electronic health record according to an embodiment of the present disclosure may filter prediction results of a model that has performed multi-stage self-training based on a predetermined criterion, and process an SQL query having the uncertainty or high probability of incorrect answer as unanswerable (null), thereby strengthening a mechanism for filtering out incorrect answers and further increasing the quality and reliability of model output.

However, effects obtainable in the present disclosure are not limited to the effects mentioned above, and other effects not mentioned may be clearly understood from following description.

### BRIEF DESCRIPTION OF DRAWINGS

FIG. 1 shows a block diagram of a computing system implementing a method for constructing a text-to-SQL (Structured Query Language) model according to an embodiment of the present disclosure.

FIG. 2 shows a block diagram of a computing device implementing a method for constructing a text-to-SQL model according to an embodiment of the present disclosure.

FIG. 3 shows a block diagram of a computing device implementing a method for constructing a text-to-SQL model according to an embodiment of the present disclosure.

FIG. 4 is a flowchart for illustrating a method for constructing a text-to-SQL model for an electronic health record according to an embodiment of the present disclosure.

FIG. 5 shows an example of an Electronic Health Record Structured Query Language (EHRSQL) query according to an embodiment of the present disclosure.

FIG. 6 shows an example of a workflow of a text-to-SQL model for an electronic health record (EHR) according to an embodiment of the present disclosure.

FIG. 7 is a flowchart for illustrating a multi-stage self-training method according to an embodiment of the present disclosure.

FIG. 8 is a conceptual diagram for illustrating a multi-stage self-training method according to an embodiment of the present disclosure.

### DETAILED DESCRIPTION OF EMBODIMENTS

Various embodiments of the present disclosure may be subjected to various transformations and may have various embodiments, and thus specific embodiments will be illustrated in the drawings and described in detail in the detailed description. Effects and features of the present disclosure and a method for achieving them will become apparent with reference to embodiments described below in detail with reference to the drawings. However, the present disclosure is not limited to the embodiments disclosed below, but may be implemented in various forms. In the following embodiments, terms such as first, second, and the like are used for the purpose of distinguishing one component from another component, rather than having a limited meaning. In addition, a singular expression includes a plural expression unless the context clearly indicates otherwise. In addition, terms such as include or have mean that features or components described herein exist, and do not preclude a possibility that one or more other features or components are added. In addition, in the drawings, sizes of the components may be exaggerated or reduced for convenience of description. For example, because a size and a thickness of each component shown in the drawings are arbitrarily indicated for convenience of description, the present disclosure is not necessarily limited to what is illustrated.

Hereinafter, the embodiments of the present disclosure will be described in detail with reference to the accompanying drawings, and the same or corresponding components will be given the same reference numerals when they are described with reference to the drawings and redundant descriptions thereof will be omitted.

### [Exemplary System Implementing Method for Constructing Text-to-SQL Model for Electronic Health Record]

Hereinafter, an exemplary system implementing a method for constructing a text-to-SQL model for an electronic health record (EHR) based on multi-stage self-training and data filtering (hereinafter, referred to as a text-to-SQL model constructing method) will be described in detail with reference to the accompanying drawings.

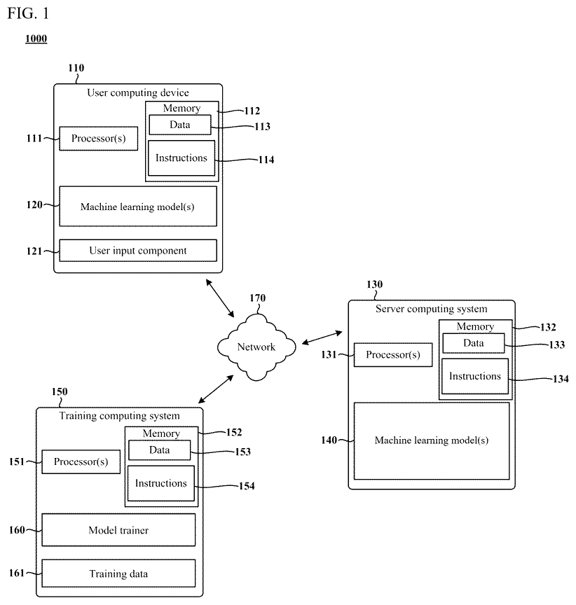

FIG. 1 shows a block diagram of a computing system implementing a method for constructing a text-to-SQL model according to an embodiment of the present disclosure.

Referring to FIG. 1, a computing system or computer 1000 implementing the method for constructing the text-to-SQL model according to an embodiment of the present disclosure may include a user computing device or user computer 110, a server computing system or server 130, and a training computing system or training computer 150, and one or more of the devices and the systems are communicatively connected to each other via a network 170.

The text-to-SQL model constructing method according to an embodiment of the present disclosure may (1) be implemented and provided locally by the user computing device 110, (2) be implemented and provided in a form of a web service by the server computing system 130 in communication with the user computing device 110, and (3) be implemented and provided by the user computing device 110 and the server computing system 130 in association or combination with each other.

In an embodiment, the user computing device 110 and/or the server computing system 130 may train machine learning models 120 and/or 140 through interaction with the training computing system 150 communicatively connected thereto via the network 170. The training computing system 150 may be a system separated from the server computing system 130 or may be included in or a part of the server computing system 130.

An artificial intelligence model may (1) be trained directly locally by the user computing device 110, (2) be trained through interaction between the server computing system 130 and the user computing device 110 via the network 170, or (3) be trained by the separate training computing system 150 using various training and learning techniques. Further, the artificial intelligence model trained by the training computing system 150 may be implemented by being transmitted to the user computing device 110 and/or the server computing system 130 via the network 170 and then being provided/updated.

In some embodiments, the training computing system 150 may be included in or a part of the server computing system 130 or the user computing device 110.

### User Computing Device 110

The user computing device 110 may include all types of computing devices or computers, such as a smart phone, a mobile phone, a device for digital broadcasting, a personal digital assistant (PDA), a portable multimedia player (PMP), a desktop, a wearable device, an embedded computing device and/or a tablet personal computer (PC).

Further, in an embodiment, the user computing device 110 may further include a server computing device or server that provides a text-to-SQL model constructing environment.

The user computing device 110 includes one or more processors 111 and one or more memories 112.

The processor 111 of the user computing device 110 may comprise, for example, but not limited to, one or more of a central processing unit (CPU), a graphics processing unit (GPU), an application specific integrated circuit (ASIC), a digital signal processor (DSP), a digital signal processing device (DSPD), a programmable logic device (PLD), a field programmable gate array (FPGA), a controller, a micro-controller, a microprocessor, and/or electrical units for performing specific or intended functions, or a plurality of processors electrically or communicationally connected to each other.

In particular, according to an embodiment, the processor 111 may be configured based on a field programmable gate array (FPGA) implementation and/or an application specific integrated circuit (ASIC), which are hardware for implementing a digital circuit.

The field programmable gate array implementation (FPGA) may refer to a flexible digital circuit that is programmable based on user needs.

In an embodiment, the field programmable gate array implementation may include a register that temporarily stores data and controls flow and timing of a signal to maintain a computation intermediate result or state information and support a synchronized operation of the FPGA, programmable logic that programs an internal computation of the FPGA so as to perform a specific function or computation as logic circuits configurable based on the user needs, an input interface that receives a signal from an external device or a sensor as a passage for receiving data from the outside of the FPGA and transmits the signal to an internal circuit, and the like.

Through a combination of the components listed above, the field programmable gate array implementation may provide flexible and various types of digital circuits.

Further, the application specific integrated circuit (ASIC) may refer to a customized integrated circuit fixedly designed for a specific purpose or for performing a specific function.

According to an embodiment, the application specific integrated circuit may include a register that stores a calculation intermediate result or state information and supports an operation of the ASIC to be rapidly processed as a small memory device that temporarily stores and manages data, a microprocessor that performs several computations or generates a control signal to adjust an operation of an entire system when necessary as a central processing device that performs control and computation inside the ASIC, an input block that, as an interface that receives data from the outside, receives data to be processed by the ASIC and transmits the received data to the inside, and receives various input data through a connection with a sensor or an external device, and the like.

Through a combination of the above components, the application specific integrated circuit may perform a task for a specific purpose in an optimized manner.

The memory 112 of the user computing device 110 may include one or more non-transitory and/or transitory computer-readable storage media, such as a Random Access Memory (RAM), a Read-Only Memory (ROM), an Electrically Erasable Programmable Read-Only Memory (EEPROM), an Erasable Programmable Read-Only Memory (EPROM), a flash memory device, a magnetic disk, and the like, and combinations thereof, and may include web storage of a server that performs a storage function of the memory on the Internet. The memory 112 may store data 113 and instructions 114 executable by or necessary for at least one processor 111 to train the artificial intelligence model and perform functional operations such as the data filtering through the artificial intelligence model.

In an embodiment, the user computing device 110 may perform various deep learning for the text-to-SQL model constructing method in association with a deep learning neural network.

The deep learning neural network according to an embodiment may include a convolution neural network (CNN), a regions with CNN features (R-CNN), a Fast R-CNN, a Faster R-CNN, a Mask R-CNN, and the like, and may include any deep learning neural network including an algorithm capable of implementing embodiments of the present disclosure. The embodiments of the present disclosure are not limited to a specific deep learning neural network.

According to an embodiment, the deep learning neural network may be directly installed in the server computing system 130, or may operate as a device separate from the server computing system 130 to perform the deep learning for the text-to-SQL model constructing method.

Further, in an embodiment, the user computing device 110 may store at least one machine learning model 120.

For example, the user computing device 110 may include various machine learning models, such as a plurality of neural networks (e.g., a deep neural network) that perform the text-to-SQL model constructing method based on structured and/or quantitative data, or other types of machine learning models including a non-linear model and/or a linear model, and may be configured in combinations thereof.

For example, the machine learning model may store linear regression, decision tree, random forest, gradient boosting pre-trained language models and/or deep learning models. The neural network may include at least one of feed-forward neural networks, recurrent neural networks (e.g., long and short-term memory recurrent neural networks), convolutional neural networks, and/or other types of neural networks.

Further, according to an embodiment, the user computing device 110 may store a model to be used in each process and a prompt template that is a basis of an input to the model to perform one or more of processes performed for the method for answering a query using a text-to-SQL model for an electronic health record through the large language model (LLM).

In an embodiment, the user computing device 110 may receive the at least one machine learning model 120 from the server computing system 130 through the network 170, store the machine learning model 120 in the memory 112, and then execute the stored machine learning model 120 by the processor 111 to perform answering a query using a text-to-SQL model for an electronic health record and the like.

In another embodiment, the user computing device 110 may perform an operation through the machine learning model 140 including at least one machine learning model 140 in association with the server computing system 130, and provide the user with the text-to-SQL model constructing method through communication of data related thereto with the outside.

For example, the user computing device 110 may perform the text-to-SQL model constructing method in a manner that the server computing system 130 provides an output for an input of the user using the machine learning model 140 via the web.

In addition, the artificial intelligence models may also be implemented in a manner that one or more of the machine learning models 120 and/or 140 are executed in the user computing device 110 and the rest of the machine learning models 120 and/or 140 are executed in the server computing system 130.

In addition, the user computing device 110 may include at least one input component 121 that senses or receives the input of the user.

For example, the user input component 121 may include a touch sensor (e.g., a touch screen and/or a touch pad) that senses a user's touch with an input medium (e.g., a finger or a stylus), an image sensor that senses a user's motion input, a microphone that senses a user's voice input, a button, a mouse, and/or a keyboard.

The image sensor may include an image processing module or an image processor. Specifically, the image sensor may process a still image or a moving image obtained by an image sensor device (e.g., a Complementary Metal-Oxide-Semiconductor (CMOS) or a Charge-Coupled Device (CCD)).

In addition, the image sensor may extract necessary information by processing a still image or a moving image obtained through the image sensor device using an image recognition process (e.g., Optical Character Recognition (OCR) or the like) and/or the image processing module, and transmit the extracted information to the processor.

In addition, the input component 121 may receive an input to an external controller (e.g., a mouse, a keyboard, and the like) based on an interface module, and may include an external output device (e.g., a speaker).

The interface module may include at least one of a wired and/or wireless headset port, an external charger port, a wired and/or wireless data port, a memory card port, a port for connecting a device having an identification module, an audio input/output (I/O) port, a video input/output (I/O) port, an earphone port, a power amplifier, a radio frequency (RF) circuit, a transceiver, and other communication circuits.

The external output device may include a display system that outputs various information related to the text-to-SQL model constructing method as a graphic image.

The display system may include at least one of a liquid crystal display (LCD), a thin film transistor-liquid crystal display (TFT LCD), an organic light-emitting diode (OLED), a flexible display, a three-dimensional (3D) display, and an e-ink display.

In one example, the user computing device 110 including the above-described components may further perform at least some of functional operations performed by the server computing system 130 to be described later.

### Server Computing System 130

The server computing system 130 may perform a series of processes for implementing the text-to-SQL model constructing method.

For example, in an embodiment, the server computing system 130 may provide the text-to-SQL model constructing method, by exchanging data necessary to cause the text-to-SQL model constructing process to be run or executed in an external device, such as the user computing device 110, with the external device.

In more detail, in an embodiment, the server computing system 130 may provide an environment in which an application may operate on the user computing device 110.

To this end, the server computing system 130 may include an application program, data, and/or instructions for the application to operate, and may transmit and receive various data based thereon to and from the external device.

In addition, the server computing system 130 includes one or more processors 131 and one or more memories 132.

The processor 131 of the server computing system 130 may comprise, for example, but not limited to, one or more of a central processing unit (CPU), a graphics processing unit (GPU), an application specific integrated circuit (ASIC), a digital signal processor (DSP), a digital signal processing device (DSPD), a programmable logic device (PLD), a field programmable gate array (FPGA), a controller, a micro-controller, a microprocessor, and/or electrical units for performing specific or intended functions, or a plurality of processors electrically or communicationally connected to each other.

In particular, according to an embodiment, the processor 131 may be configured based on a field programmable gate array (FPGA) implementation and/or an application specific integrated circuit (ASIC), which are hardware for implementing a digital circuit.

Further, the memory 132 may include one or more non-transitory and/or transitory computer-readable storage media, such as a RAM, a ROM, an EEPROM, an EPROM, a flash memory device, a magnetic disk, and the like, and combinations thereof. The memory 132 may store data 133 and instructions 134 executable by or necessary for the processor 131 to train the artificial intelligence model or perform functional operations such as executing a method for answering a query using a text-to-SQL model for an electronic health record through the artificial intelligence model.

In an embodiment, the server computing system 130 may include at least one computing device or computer. For example, the server computing system 130 may be configured to operate the plurality of computing devices based on a sequential computing architecture, a parallel computing architecture, or a combination thereof. Further, the server computing system 130 may include the plurality of computing devices or computers communicationally connected to each other via the network 170.

Further, the server computing system 130 may store at least one machine learning model 140. For example, the server computing system 130 may include neural networks and/or other multi-layer non-linear models as the machine learning models 140. Exemplary neural networks may include, for instance, but not limited to, a feed forward neural network, a deep neural network, a recurrent neural network, and a convolutional neural network.

In an embodiment, the server computing system 130 may further include a data storage computing system (hereinafter, referred to as a data storage) that is storage for continuously storing and managing raw data that is a basis of the text-to-SQL model constructing method.

The data storage may include various types of data storage, ranging from a file system to cloud storage. For example, the data storage may include at least one database among a relational database that uses a structured query language (SQL) to define and manipulate data, an NoSQL database that is designed for flexibility and scalability to process unstructured and semi-structured data, a data warehouse that is optimized for query and analysis by centralizing a large amount of data from multiple sources as a system used for reporting and data analysis, a data warehouse that stores a large amount of raw data in basic formats such as structured data, semi-structured data, and unstructured data, and a local storage device or a network attached storage (NAS) that stores data in a file in a format generally accessible by a computer operating system.

### Training Computing System 150

The training computing system 150 includes one or more processors 151 and one or more memories 152.

The processor 151 of the training computing system 150 may comprise, for example, but not limited to, one or more of a central processing unit (CPU), a graphics processing unit (GPU), an application specific integrated circuit (ASIC), a digital signal processor (DSP), a digital signal processing device (DSPD), a programmable logic device (PLD), a field programmable gate array (FPGA), a controller, a micro-controller, a microprocessor, and/or electrical units for performing specific or intended functions, or a plurality of processors electrically or communicationally connected to each other.

In particular, according to an embodiment, the processor 151 may be configured based on a field programmable gate array (FPGA) implementation and/or an application specific integrated circuit (ASIC), which are hardware for implementing a digital circuit. A detailed description thereof will be omitted by applying the description of the FPGA and the ASIC above.

Further, the memory 152 may include one or more non-transitory/transitory computer-readable storage media, such as a RAM, a ROM, an EEPROM, an EPROM, a flash memory device, a magnetic disk, and the like, and combinations thereof. The memory 152 may store data 153 and instructions 154 executable by or necessary for the processor 151 to perform training or the like of the artificial intelligence model.

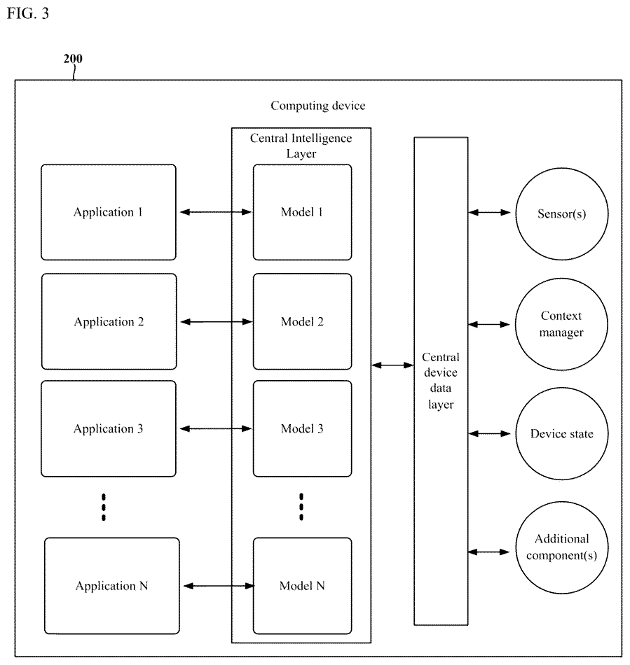

For example, the training computing system 150 may include a model trainer 160 that uses various training or learning techniques, such as backpropagation of an error (based on a framework shown in FIG. 3), to train the machine learning models 120 and/or 140 stored in the user computing device 110 and/or the server computing system 130.

The model trainer 160 may perform an update of one or more parameters of the machine learning models 120 and/or 140 for the text-to-SQL model constructing method based on a defined loss function in a backpropagation manner.

In some implementations, performing the backpropagation of the error may include performing truncated backpropagation through time. The model trainer 160 may perform a number of generalization techniques (e.g., weight reduction, dropout, and/or knowledge distillation) to improve generalization capability of the trained machine learning models 120 and/or 140.

Further, the model trainer 160 may train the machine learning models 120 and/or 140 based on a series of training data 161. The training data 161 may include, for example, different forms of data, such as images, audio samples, and/or text. Examples of image types that may be used may include a video frame, a LiDAR point cloud, an X-ray image, a computed tomography scan, a hyperspectral image, and/or images of various other forms.

The training data 161 may be provided by the user computing device 110 and/or the server computing system 130. When a training computing device trains the machine learning models 120 and/or 140 for specific data of the user computing device 110, the machine learning models 120 and/or 140 may be characterized as personalized models.

Further, the model trainer 160 includes computer logic utilized to provide desired functionality.

In addition, the model trainer 160 may be implemented as hardware, firmware, and/or software that controls a general-purpose processor. In one implementation, the model trainer 160 may include a program file stored in a storage device, and may be loaded into or stored in the memory 152 and executed by the one or more processors 151. In another implementation, the model trainer 160 includes one or more sets of the computer-executable data 153 and the instructions 154 stored in a tangible computer-readable storage medium such as a RAM, a hard disk, or an optical or magnetic medium.

The network 170 includes a 3rd generation partnership project (3GPP) network, a long term evolution (LTE) network, a world interoperability for microwave access (WIMAX) network, the Internet, a local area network (LAN), a wireless local area network (Wireless LAN), a wide area network (WAN), a personal area network (PAN), a Bluetooth network, a satellite broadcasting network, an analog broadcasting network, a digital multimedia broadcasting (DMB) network, and/or the like, but is not limited thereto.

In general, communication over the network 170 may be performed using any type of wired and/or wireless connection, via various communication protocols (e.g., TCP/IP, HTTP, SMTP, and/or FTP), encodings or formats (e.g., HTML and/or XML), and/or security schema (e.g., VPN, secure HTTP and/or SSL).

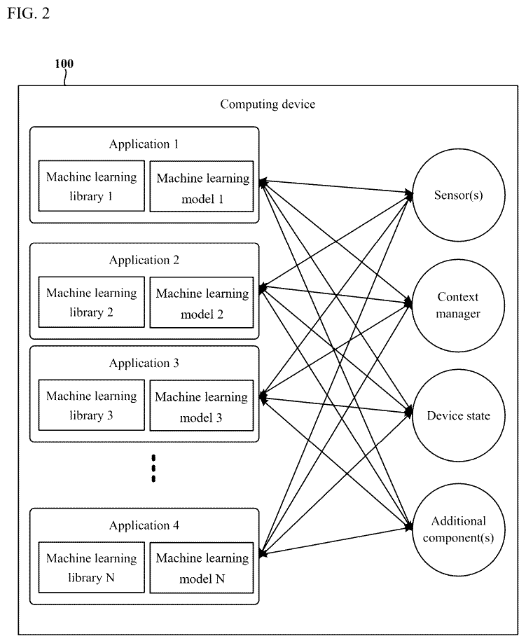

FIG. 2 shows a block diagram of a computing device implementing a method for constructing a text-to-SQL model according to an embodiment of the present disclosure.

Referring to FIG. 2, a computing device 100 included in each of the user computing device 110, the server computing system 130, and the training computing system 150 includes multiple applications (e.g., Applications 1 to N). Each application may include a machine learning library and one or more machine learning models. For example, the application may include an application for image processing (e.g., detection, classification, and/or segmentation) application, a text messaging application, an email application, a dictation application, a virtual keyboard application, a browser application, and/or a chat-bot application.

In an embodiment, the computing device 100 may include the model trainer 160 for training the artificial intelligence model, and may store and operate the trained artificial intelligence model, thereby providing output data based on predetermined input data (e.g., a natural language query or the like).

Each application of the computing device 100 may be, for example, in communication with a number of other components of the computing device 100, such as at least one sensor, a context manager, a device state component, and/or additional components. In one embodiment, each application may communicate with each device component using an Application Programming Interface (API) (e.g., a public API). In one embodiment, the API used by each application may be specific to a corresponding application.

FIG. 3 shows a block diagram of a computing device implementing a method for constructing a text-to-SQL model according to an embodiment of the present disclosure.

Referring to FIG. 3, the computing device 200 includes multiple applications (e.g., Applications 1 to N). Each application may communicate with a central intelligence layer. For example, the applications may include an image processing application, a text message application, an email application, a dictation application, a virtual keyboard application, and/or a browser application. In an embodiment, each application may communicate the central intelligence layer (and a model stored therein) using an API (e.g., a common API across all applications).

The central intelligence layer may include multiple machine learning models. For example, as shown in FIG. 3, one or more of the machine learning models may be provided for respective applications and managed by the central intelligence layer. In another implementation, two or more applications may share a single machine learning model. For example, in some implementations, the central intelligence layer may provide a single model for all applications. In some implementations, the central intelligence layer may be included in an operating system of the computing device 200 or may alternatively be implemented separately.

The central intelligence layer may communicate with a central device data layer. The central device data layer may be centralized data storage for the computing device 200. For example, as shown in FIG. 3, the central device data layer may communicate with a number of other components of the computing device 200, such as one or more sensors, a context manager, a device state component, and/or additional components. In some implementations, the central device data layer may be in communication with each device component using an API (e.g., a private API).

The technology described herein may refer to a server, a database, software applications, and other computer-based systems as well as actions taken and information transmitted to or transmitted from the system. It will be appreciated that inherent flexibility of computer-based systems allows for a wide range of possible configurations, combinations and division of work, and functionality between and from components. For example, the processes described herein may be implemented using a single device or component or multiple devices or components operating in combination. The database and the applications may be implemented in a single system or a distributed system across multiple systems. Distributed components may operate sequentially or in parallel.

### [Text-to-Structured Query Language (SQL) Model Construction Method for Electronic Health Record]

Hereinafter, a method for constructing a text-to-SQL model for an electronic health record (EHR) based on multi-stage self-training and data filtering according to an embodiment of the present disclosure will be described in detail with reference to the accompanying drawings.

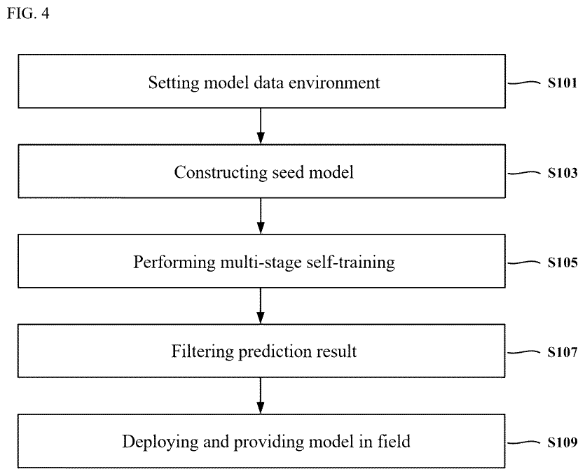

FIG. 4 is a flowchart for illustrating a method for constructing a text-to-SQL model for an electronic health record according to an embodiment of the present disclosure.

Referring to FIG. 4, the text-to-SQL model constructing method according to an embodiment of the present disclosure may include step S101 of setting a model data environment, step S103 of constructing a seed model, step S105 of performing a multi-stage self-training, step S107 of performing a prediction result filtering, and step S109 of deploying and providing a model in the field.

At step S101, the computing system 1000 according to an embodiment of the present disclosure may set the model data environment.

For instance, in an embodiment, the computing system 1000 may (1) construct an EHR database.

The EHR database according to an embodiment may include a relational database configured to store and manage at least one electronic health record (EHR) and the like.

The electronic health record (EHR) may comprise a comprehensive relational database configured to record an overall medical history including diagnosis, hospitalization, treatment, discharge, medical administration, and the like of a specific patient.

The electronic health record (EHR) may generally be implemented in a form accessible through a query language such as structured query language (SQL). Therefore, only users with technical expertise in handling queries may easily secure their access thereto.

In addition, in an embodiment, the computing system 1000 may (2) construct an electronic health record structured query language (EHRSQL) database.

The EHRSQL database according to an embodiment may be configured to store and manage at least one EHRSQL and the like.

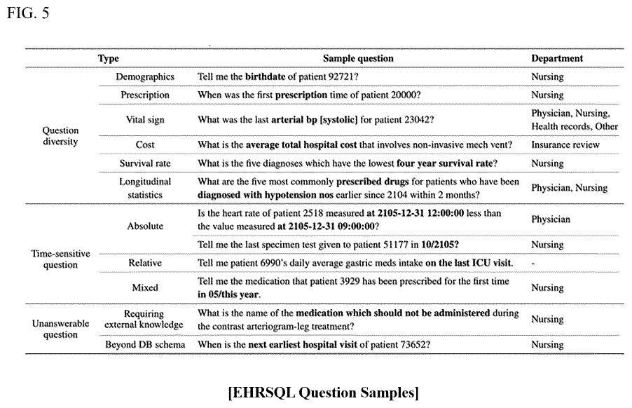

For example, the EHRSQL may be a professional text-to-SQL data set designed to improve accessibility to the electronic health record (EHR) by converting a specific natural language query into an SQL query (see FIG. 5).

Accordingly, the EHRSQL may be implemented in a shape reflecting EHR database schema.

In an embodiment, the computing system 1000 may classify data in the EHRSQL database into a training data set, a verification data set, and/or a test data set.

Here, the training data set may refer to a data set used for the model training.

In an embodiment, the training data set may include data in which a ground-truth SQL query based on the electronic health record (EHR) is paired with a corresponding natural language query written by medical experts in an actual clinical environment (hereinafter referred to as “answerable query data”) and data in which null data is paired with a natural language query (hereinafter referred to as “unanswerable query data”).

In addition, in an embodiment, the training data set may further include additional unanswerable query data obtained through the self-training to be described later.

In one example, the verification data set may refer to a data set used for model verification such as intermediate inspection and parameter tuning for a model performance.

In addition, the test data set may refer to a data set used for final performance evaluation of the model.

In an embodiment, the verification data set and/or the test data set may be configured to include a predetermined natural language query data set.

At step S103, the computing system 1000 according to an embodiment of the present disclosure may construct a seed model.

For example, in an embodiment, the computing system 1000 may construct the seed model based on a pre-trained large language model (LLM) and/or a specific text-to-SQL model. The seed model may be, for example, but not limited to, a base model configured to provide a starting point of the model learning.

The computing system 1000 may select at least one of various models including the pre-trained large language model (LLM) and/or the specific text-to-SQL model to construct the seed model.

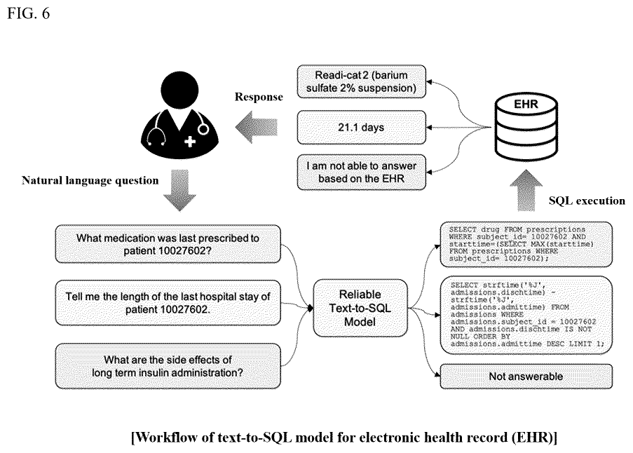

FIG. 6 shows an example of a workflow of a text-to-SQL model for an electronic health record (EHR) according to an embodiment of the present disclosure.

Referring to FIG. 6, the text-to-SQL model may comprise an artificial intelligence model that incorporates natural language processing (NLP) to convert a natural language query into a database query (SQL) and a database (DB) technology.

The text-to-SQL model may generate and output the SQL query corresponding to the natural language query by reflecting schema information of the related database (i.e., database schema).

The database schema, as a design diagram defining a structure of the database, may be logical design information describing a structure, constraints, and/or relationships of data to be stored in the database.

In other words, the database schema may define what information the database stores and interacts with, and may be information including definitions of database objects, such as a table, a field, a data type, a primary/foreign key, an index, a view, and/or constraints.

For example, when the natural language query such as “How many patients diagnosed with diabetes among patients 65 years of age or older for the past 6 months?” is input, the text-to-SQL model may output the SQL query such as “SELECT COUNT(*) FROM patient_records WHERE age>=65 AND diagnosis=‘diabetes’ AND visit_date>=DATE_SUB(CURDATE( ), INTERVAL 6 MONTH);”.

The text-to-SQL model may support a user to easily and intuitively perform the database query through the natural language even when the user is not proficient in the SQL syntax.

However, in an existing text-to-SQL model, when the structure of the database is complicated or the natural language query is ambiguous, a possibility of error occurrence, such as propagation of incorrect information or provision of a forcedly generated answer, may increase.

Accordingly, the computing system 1000 according to an embodiment of the present disclosure implementing a text-to-SQL model constructing mechanism for the electronic health record (HER) may accurately detect an unanswerable query or an uncertain prediction by performing the multi-stage self-training and the data filtering to be described later, and reject to provide an answer to the query or performing additional confirmation.

At step S105, the computing system 1000 according to an embodiment of the present disclosure may perform the multi-stage self-training.

The multi-stage self-training according to an embodiment may perform semi-supervised learning in which a model performs self-prediction on data in which a label is absent and re-trains itself.

As described above, to construct the text-to-SQL model for the electronic health record (EHR) capable of accurately detecting an unanswerable query or uncertain prediction and rejecting to provide an answer thereto or performing additional confirmation, it may be advantageous to increase the amount of training data associated with unanswerable queries.

However, because it usually takes a lot of cost such as money and time to manually discover and label the unanswerable queries (e.g., a query for which a ground-truth SQL query does not exist or external knowledge is required), there is a practical limit in constructing a large-scale data set.

Therefore, the computing system 1000 according to an embodiment of the present disclosure may perform the multi-stage self-training, which automatically identifies a query that is not able to be answered based on a prediction generated by the model itself (i.e., a pseudo-label) and reflects the same to the learning again.

Accordingly, the computing system 1000 according to an embodiment may improve a model's ability to distinguish between answerable query data and unanswerable query data, and as a result, may construct the text-to-SQL model with an improved performance that outputs a more reliable SQL query.

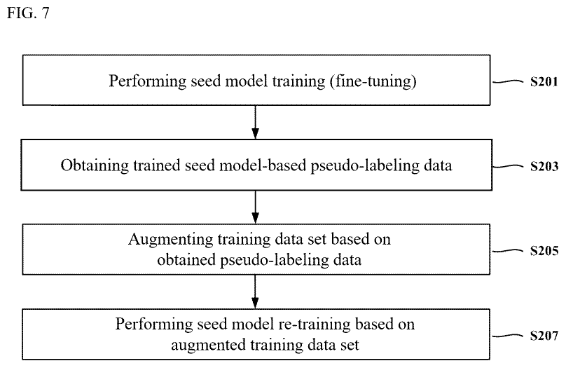

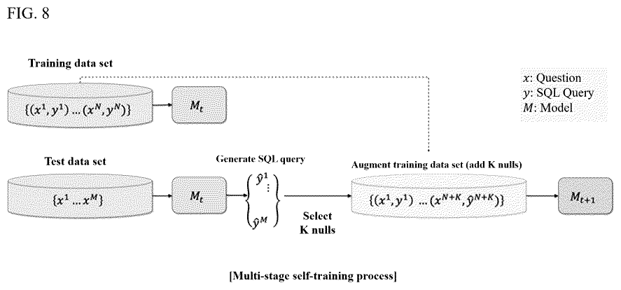

FIG. 7 is a flowchart for illustrating a multi-stage self-training method according to an embodiment of the present disclosure, and FIG. 8 is a conceptual diagram for illustrating a multi-stage self-training method according to an embodiment of the present disclosure.

Referring to FIGS. 7 and 8, at step S201, the computing system 1000 according to an embodiment may perform seed model training (fine-tuning).

For example, in an embodiment, the computing system 1000 may perform training (e.g., fine tuning) on a seed model using a training data set in the EHRSQL database described above.

The training data set according to an embodiment may include data in which a ground-truth SQL query based on an electronic health record (EHR) is matched to or paired with a natural language query written by medical experts in an actual clinical environment (i.e., answerable query data) and data in which null data is matched to a natural language query (i.e., unanswerable query data).

Accordingly, the computing system 1000 may implement the seed model configured to understand the data schema structure of the electronic health record (EHR) through the seed model training based on the training data set.

At step S203, the computing system 1000 according to an embodiment may obtain the trained seed model-based pseudo-labeling data.

Here, the pseudo-labeling data according to an embodiment may comprise data in which a pseudo-label in a form corresponding to unanswerable is assigned to a natural language query predicted to be unanswerable by the fine-tuned seed model.

For instance, in an embodiment, the computing system 1000 may obtain output data (e.g., SQL query or null data) based on each sample of test data set using the trained seed model (hereinafter, referred to as a fine-tuned seed model) and the test data set in the EHRSQL database described above.

Specifically, in an embodiment, the computing system 1000 may input each sample of the test data set (hereinafter, test sample data) to the fine-tuned seed model.

Then, the fine-tuned seed model according to an embodiment may perform a prediction process based on the input test sample data to generate output data (e.g., a prediction value) based thereon.

Then, the seed fine-tuned model may provide the generated output data to the computing system 1000.

Thus, in an embodiment, the computing system 1000 may obtain the output data for each test sample data based on the fine-tuned seed model.

In an embodiment, when the obtained output data is the null data (i.e., when the fine-tuned seed model predicts the natural language query data as unanswerable), the computing system 1000 may map the pseudo-label having a form corresponding to unanswerable to the corresponding natural language query data.

Thus, in an embodiment, the computing system 1000 may obtain at least one pseudo-labeling data in which the natural language query data predicted as unanswerable and the pseudo-label in the form corresponding thereto are paired or combined with each other.

At step S205, the computing system 1000 according to an embodiment may augment the training data set based on the obtained pseudo-labeling data.

For instance, in an embodiment, the computing system 1000 may augment the training data set by adding at least one (e.g., K (K>1)) pseudo-labeling data to the training data set described above.

In other words, the computing system 1000 may pair or combine at least one pseudo-labeling data with an initial training data set to obtain a training data set further including new unanswerable query data (hereinafter, referred to as an extended training data set).

At step S207, the computing system 1000 according to an embodiment may perform seed model re-training based on the augmented training data set.

For example, in an embodiment, the computing system 1000 may perform re-training of the seed model (i.e., additional training of the fine-tuned seed model) using the extended training data set as described above.

As such, in an embodiment, the computing system 1000 may construct a model that has learned more cases of the unanswerable query data, thereby improving the corresponding model's ability to distinguish between the answerable query and the unanswerable query, and enhancing accuracy of refusal to answer (null) predictions for the unanswerable query.

According to an embodiment, the computing system 1000 may verify performance of the re-trained seed model (hereinafter, referred to as the re-trained seed model) using the verification data set in the EHRSQL database described above.

Specifically, in an embodiment, the computing system 1000 may examine the performance of the re-trained seed model based on the verification data set using a predetermined evaluation index.

In detail, in an embodiment, the computing system 1000 may input each sample of the verification data set (hereinafter, verification sample data) to the re-trained seed model.

Then, the re-trained seed model according to an embodiment may perform a prediction process based on the input verification sample data to generate output data (e.g., a prediction value) based thereon.

Then, the re-trained seed model may provide the generated output data to the computing system 1000.

Thus, in an embodiment, the computing system 1000 may obtain the output data for each verification sample data based on the re-trained seed model.

In addition, in an embodiment, the computing system 1000 may perform a verification process for the re-trained seed model based on the obtained output data and the predetermined evaluation index.

In an embodiment, the computing system 1000 may measure and monitor a reliability score (RS) based on the output data of the seed re-training model.

For reference, referring to Equation 1 below, the reliability score (RS) may be an evaluation index that measures a model's ability to refrain from responding to an unanswerable query ($Q_{ans}$) while generating a correct SQL query for an answerable query ($Q_{una}$).

$${ϕ}_{c} ( x ) = \{ \begin{matrix}1 & if x ∈ {𝔔}_{ans} ; g ( x ) = 1 ; Acc ( x ) = 1 \\ 0 & if x ∈ {𝔔}_{ans} ; g ( x ) = 0 , \\ - c & if x ∈ {𝔔}_{ans} ; g ( x ) = 1 ; Acc ( x ) = 0 \\ - c & if x ∈ {𝔔}_{una} ; g ( x ) = 1 , \\ 1 & if x ∈ {𝔔}_{una} ; g ( x ) = 0.\end{matrix} \qquad [ Equation 1 ]$$

Here, ‘g(x)’ in Equation 1 may indicate whether the model generates SQL (g(x)=1) or not (g(x)=0) for a given query, ‘Acc(x)’ may indicate whether an output of the model matches an actual value (i.e., execution accuracy), and ‘c’ may indicate an imposed penalty weight.

The reliability score (RS) may be calculated in a way that the score is increased when the accurate SQL query is generated for the answerable query or a response is rejected for the unanswerable query, and the score is decreased when an incorrect SQL query is generated for the answerable query or an SQL query generation is tried for the unanswerable query.

That is, the reliability score (RS) may be calculated based on whether the model generates or refrains from generating the SQL query and an execution accuracy ($Acc$).

In addition, according to an embodiment, the computing system 1000 that has verified the performance of the re-trained seed model using the evaluation index may adjust hyperparameters (e.g., a learning rate and/or a batch size) of the re-trained seed model based on the verification result.

In addition, according to an embodiment, the computing system 1000 may determine whether to perform the additional training on the re-trained seed model based on the verification result.

In an embodiment, the computing system 1000 may repeatedly perform steps S201 to S207 described above until the reliability score (RS) becomes equal to or greater than a predetermined threshold.

As described above, in an embodiment, the computing system 1000 may support model performance improvement resulted from the additional training based on the extended data set by automatically assigning the pseudo-label to the unanswerable query through the multi-stage self-training technique and using the pseudo-label for the training again.

Accordingly, the computing system 1000 may allow the text-to-SQL model for the electronic health record (EHR) to more accurately detect the unanswerable query or the uncertain prediction, and to refuse to provide the answer therefor or perform the additional verification procedure.

As a result, the computing system 1000 may provide the text-to-SQL model with the improved performance that generates the SQL query of higher accuracy and reliability more reliably in the actual medical environment.

Referring back to FIG. 4, at step S107, the computing system 1000 according to an embodiment of the present disclosure may perform prediction result filtering.

For example, in an embodiment, to further guarantee a quality of the prediction result (e.g., a prediction value) of the seed model that has performed the multi-stage self-training (i.e., the re-trained seed model), the computing system 1000 may perform a prediction result filtering process of refining the output data of the re-trained seed model based on a predetermined filtering criterion.

In detail, in an embodiment, the computing system 1000 may perform (1) maximum entropy filtering on the output data of the re-trained seed model.

Specifically, in an embodiment, the computing system 1000 may calculate a probability distribution for the SQL query based on the prediction result of the re-trained seed model, and may calculate an entropy at a query or a token level based on the probability distribution.

For reference, the entropy is an indicator representing a degree of uncertainty in the probability distribution. For example, when probabilities for possible values of a random variable X are evenly distributed (i.e., when it is difficult to predict which value will come out), the entropy may increase, whereas when the probability is concentrated on a specific value (i.e., when a result is relatively certain), the entropy may decrease.

In this regard, in an embodiment, when the calculated entropy is equal to or greater than a predetermined threshold (i.e., when there is uncertainty equal to or greater than a predetermined threshold), the computing system 1000 may process the corresponding SQL query as the unanswerable (null) data or filter-out the corresponding SQL query as a target requiring additional verification.

As such, in an embodiment, the computing system 1000 may increase the accuracy of the answer and prevent the propagation of incorrect information by filtering the SQL query generated by the model in an uncertain manner.

In addition, in an embodiment, the computing system 1000 may perform (2) execution-based filtering on the output data of the re-trained seed model.

For example, in an embodiment, the computing system 1000 may actually execute the SQL query based on the prediction result of the re-trained seed model in an EHR database environment, or may execute the same in a sample database and/or a dedicated test environment.

In this regard, in an embodiment, when a predetermined error (e.g., an SQL syntax error, a table-column non-existence error, and/or a valid value (i.e., an intended result) absence error) is detected in the process of executing the SQL query, the computing system 1000 may process the corresponding SQL query as the unanswerable (null) data or filter-out the target requiring the additional verification.

As such, in an embodiment, the computing system 1000 may filter SQL queries that contain syntactic and/or semantic issues, taking into account actual execution results, to provide SQL queries whose actual validity has been verified, thereby further improving the reliability of the SQL queries.

As described above, in an embodiment, the computing system 1000 may filter the prediction results of the seed model that has performed the multi-stage self-training (i.e., the re-trained seed model) based on the predetermined criterion, and process the SQL query having an uncertainty or a high probability of incorrect answer as unanswerable (null), thereby strengthening a mechanism for filtering out the incorrect answers and further increasing the quality and reliability of the SQL query.

At step S109, the computing system 1000 according to an embodiment of the present disclosure may deploy and provide the model in the field.

For example, in an embodiment, the computing system 1000 may deploy and provide the text-to-SQL model for the electronic health record (EHR) that performs the multi-stage self-training and the prediction result filtering as described above in the actual field in a predetermined manner.

In an embodiment, the computing system 1000 may provide the above-described text-to-SQL model in association with an application service operating in a predetermined medical field.

In this regard, according to an embodiment, the computing system 1000 may collect feedback information on the provided text-to-SQL model.

For example, the computing system 1000 may collect the feedback information such as user performance evaluation for the model, new query type, and/or new medical data.

Then, the computing system 1000 may support updating of the text-to-SQL model based on the collected feedback information.

As described above, in an embodiment of the present disclosure, the computing system 1000 may provide the text-to-SQL model for the electronic health record (EHR) to which the multi-stage self-training and multi-filtering (in an embodiment, maximum entropy filtering and the execution-based filtering) strategy are applied.

Accordingly, the computing system 1000 may implement a text-to-SQL system that provides high accessibility, accuracy, and reliability to the electronic health record (EHR) environment.

Specifically, the computing system 1000 may precisely distinguish between the answerable query and the unanswerable query through a combination of the multi-stage self-training and the filtering strategy, and at the same time, convert the answerable query into the accurate SQL query and process the unanswerable query as unanswerable (null), thereby supporting the user (in an embodiment, a medical staff or the like) to safely and accurately retrieve the electronic health record (EHR) data without the separate SQL knowledge, and further increasing accuracy of medical decision making and patient treatment efficiency by minimizing a risk of the propagation of the incorrect information.

As a result, the computing system 1000 according to an embodiment of the present disclosure may provide a text-to-SQL solution that lowers a barrier to entry caused by a need to acquire specialized knowledge and has enhanced reliability, thereby providing a practical solution for realizing a stable and rapidly-implemented query answering in the electronic health record (EHR) environment and reducing a gap between medical technology and clinical practice.

As described above, the method and the system for constructing the text-to-SQL model for the electronic health record (EHR) according to an embodiment of the present disclosure may construct the text-to-SQL model for the electronic health record (EHR) based on the multi-stage self-training and the data filtering, thereby allowing the user to safely retrieve the accurate electronic health record (EHR) data without the separate SQL knowledge, and thus, minimizing the propagation of the incorrect information and further improving the accuracy of the medical decision making and the patient treatment efficiency.

That is, the method and the system for constructing the text-to-SQL model for the electronic health record (EHR) according to an embodiment of the present disclosure may provide the text-to-SQL solution that lowers the barrier to entry caused by the need to acquire the specialized knowledge and has the enhanced reliability, thereby providing the practical solution for realizing the stable and rapidly-implemented query answering in the electronic health record (EHR) environment and reducing the gap between the medical technology and the clinical practice.

In addition, the method and the system for constructing the text-to-SQL model for the electronic health record (EHR) according to an embodiment of the present disclosure may automatically assign the pseudo-label for the unanswerable query through the multi-stage self-training technique and support the additional training based on the extended data set including the pseudo-label, thereby providing the text-to-SQL model with the improved performance that stably generates the SQL query with the higher accuracy and reliability in the actual medical environment.

Accordingly, the method and the system for constructing the text-to-SQL model for the electronic health record (EHR) according to an embodiment of the present disclosure may allow the text-to-SQL model for the electronic health record (EHR) to more accurately detect the unanswerable query or the uncertain prediction, and to refuse to provide the answer therefor or perform the additional verification procedure.

In addition, the method and the system for constructing the text-to-SQL model for the electronic health record (EHR) according to an embodiment of the present disclosure may filter the prediction results of the model that has performed the multi-stage self-training based on the predetermined criterion, and process the SQL query having the uncertainty or the high probability of incorrect answer as unanswerable (null), thereby strengthening the mechanism for filtering out the incorrect answers and further increasing the quality and the reliability of the model output.

The embodiments of the present disclosure described above may be implemented in a form of program instructions that may be executed through various computer components and recorded in a computer-readable recording medium. The computer-readable recording medium may include program instructions, data files, data structures, and the like alone or in combination. The program instructions recorded in the computer-readable recording medium may be specially designed and configured for various embodiments of the present disclosure or may be known and available to those skilled in a field of computer software. Examples of the computer-readable recording media include magnetic media such as a hard disk, a floppy disks, and a magnetic tape, optical recording media such as a CD-ROM and a DVD, magneto-optical media such as a floptical disk, and hardware devices specially configured to store and execute the program instructions such as a ROM, a RAM, a flash memory, and the like. Examples of the program instructions include not only machine language codes such as those generated by a compiler, but also high-level language codes that may be executed by a computer using an interpreter or the like. The hardware device may be changed to one or more software modules to perform the processing according to the present disclosure, and vice versa.

The specific executions described in the present disclosure are merely embodiments, and do not limit the scope of the present disclosure in any way. For the sake of brevity of the present document, a description of existing electronic components, control systems, software, and other functional aspects of the above-mentioned systems may be omitted. In addition, connection or connection members of lines between the components illustrated in the drawings exemplarily represent functional connections and/or physical or circuit connections, and may be represented as various functional connections, physical connections, or circuit connections that are replaceable or additional in an actual device. Furthermore, unless specifically stated otherwise with terms such as “essential” or “critical”, components described herein may not be strictly required for the application of the present disclosure.

Although the detailed description of the present disclosure has been provided with reference to preferred embodiments, it will be understood by those skilled in the art that the present disclosure may be variously modified and changed without departing from the spirit and scope of the disclosure as defined in the appended claims. Accordingly, the technical scope of the present disclosure should not be limited to the contents described in the detailed description but should be defined by the claims.

Some embodiments of the present disclosure relate to a method and system for constructing a Text-to-SQL model for electronic health records, and thus, have industrial applicability, as it can be utilized in the artificial intelligence industry.

What is claimed is:

1. A computerized method comprising:
receiving a natural language query from a user;
generating an output including one of a structured query language (SQL) query and a null value for the natural language query by a text-to-SQL model for an electronic health record, the text-to-SQL model configured to process the natural language query; and
outputting a response to the natural language query based on the output including one of the SQL query and the null value for the natural language query.

2. The computerized method of claim 1, wherein the outputting of the response to the natural language query includes outputting data retrieved from an electronic health record database configured to store the electronic health record based on the SQL query when the output includes the SQL query.

3. The computerized method of claim 1, wherein the outputting of the response to the natural language query includes, when the output value includes the null value, outputting at least one of a query rejection response or a response suggesting additional confirmation to the user.

4. The computerized method of claim 3, wherein the response suggesting the additional confirmation to the user includes at least one of a request to rephrase the query, a suggestion to search external information, and a guidance to contact a system administrator.

5. The computerized method of claim 1, wherein the generating of the output including one of the SQL query and the null value for the natural language query includes generating the output using the text-to-SQL model that has performed one of multi-stage self-training of re-training the text-to-SQL model itself based on an output generated by the text-to-SQL itself after initial training, and prediction result filtering.

6. The computerized method of claim 5, wherein the multi-stage self-training includes:
setting a model data environment including the electronic health record database storing the electronic health record and an electronic health record SQL database storing an electronic health record SQL;
training a seed model configured to process a predetermined natural language query based on the electronic health record database and an electronic health record SQL database-based training data set;
obtaining pseudo-labeling data comprising data assigned with a predetermined pseudo-label, based on the trained seed model;
augmenting the training data set based on the pseudo-labeling data; and
re-training the trained seed model based on the augmented training data set.

7. The computerized method of claim 6, wherein the electronic health record SQL includes a predetermined SQL query based on the predetermined natural language query and a schema of the electronic health record database.

8. The computerized method of claim 7, wherein the electronic health record SQL database includes the training data set used for model training and a test data set used for model evaluation.

9. The computerized method of claim 8, wherein the obtaining of the pseudo-labeling data includes obtaining an output value for each of test sample data included in the test data set using the trained seed model.

10. The computerized method of claim 9, wherein the obtaining of the pseudo-labeling data includes, when the obtained output value is null, mapping a pseudo-label having a form corresponding to null to test sample data having an output of null.

11. The computerized method of claim 8, wherein the prediction result filtering includes at least one of maximum entropy filtering or execution-based filtering.

12. A system comprising:
at least one memory configured to store instructions that are executable; and
at least one processor configured to execute one or more of the instructions to perform operations comprising:
receiving a natural language query from a user;
generating an output including one of a structured query language (SQL) query and a null value for the natural language query by a text-to-SQL model for an electronic health record, the text-to-SQL model configured to process the natural language query; and
outputting a response to the natural language query based on the output including one of the SQL query and the null value for the natural language query.

13. The system of claim 12, further comprising:
a plurality of neurons configured in an array including at least one register, at least one programmable logic, and at least one input interface;
a plurality of synaptic circuits configured to store synaptic weights for adjusting connection strengths between the plurality of neurons; and
at least one routing network configured to control a data flow between the plurality of neurons,
wherein each of the plurality of neurons includes a field programmable gate array (FPGA) for a predetermined artificial neural network connected to at least other neuron through the at least one routing network to set a transmission path of the synaptic weights.

14. The system of claim 12, further comprising:
a plurality of neurons organized in an array including at least one register, at least one microprocessor, and at least one input; and
a plurality of synaptic circuits configured to store synaptic weights for adjusting connection strengths among the plurality of neurons,
wherein each of the plurality of neurons includes an application specific integrated circuit (ASIC) for a predetermined artificial neural network connected to at least other neuron through one of the plurality of synaptic circuits.
